(19) United States
(12) Patent Application Publication
(10) Pub. No.: US 2009/0209493 A1
(43) Pub. Date: Aug. 20, 2009

(54) COMBINATION THERAPY COMPRISING A BISPHOSPHONATE AND A HMG-COA REDUCTASE INHIBITOR

(76) Inventors: Cindy Maree Baulch-Brown, South Morang (AU); Andrew Spencer, South Yarra (AU)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 12/431,347

(22) Filed: Apr. 28, 2009

Related U.S. Application Data
(62) Division of application No. 10/526,282, filed on Feb. 16, 2006.

(30) Foreign Application Priority Data
Sep. 9, 2002 (GB) 0220885.8

(51) Int. Cl. A61K 31/675; A61P 35/00

(52) U.S. Cl. 514/94

(57) ABSTRACT

A pharmaceutical composition for treatment of malignancies, in particular multiple myeloma (MM), comprises in combination a bisphosphonate, e.g. zoledronic acid or a salt or ester, and an HMG-CoA reductase inhibitor for simultaneous, sequential or separate use. Also provided is a method of treating a patient suffering from a malignant disease comprising administering to the patient an effective amount of a bisphosphonate and an effective amount of an HMG-CoA reductase inhibitor.

This invention relates to bisphosphonates, in particular to new pharmaceuticals uses of, and compositions containing, bisphosphonates.

Bisphosphonates are widely used to inhibit osteoclast activity in a variety of both benign and malignant diseases, which involve excessive or inappropriate bone resorption. These pyrophosphate analogs not only reduce the occurrence of skeletal related events but they also provide patients with clinical benefit and improve survival. Bisphosphonates are able to prevent bone resorption in vivo; the therapeutic efficacy of bisphosphonates has been demonstrated in the treatment of osteoporosis, osteopenia, Paget's disease of bone, tumour-induced hypercalcemia (TIH) and, more recently, bone metastases (BM) and multiple myeloma (MM) (for review see Fleisch H 1997 Bisphosphonates clinical. In Bisphosphonates in Bone Disease. From the Laboratory to the Patient. Eds: The Parthenon Publishing Group, New York/London pp 68-163). The mechanisms by which bisphosphonates inhibit bone resorption are still not completely understood and seem to vary according to the bisphosphonates studied. Bisphosphonates have been shown to bind strongly to the hydroxyapatite crystals of bone, to reduce bone turn-over and resorption, to decrease the levels of hydroxyproline or alkaline phosphatase in the blood, and in addition to inhibit the formation, recruitment, activation and the activity of osteoclasts.

Recent studies have also shown that some bisphosphonates may have a direct effect on tumour cells. Thus for example it has been found that relatively high concentrations of bisphosphonates, including zoledronate, induce apoptosis of breast and prostate carcinoma and myeloma cells in vitro (Senaratne et al. Br. J. Cancer, 82: 1459-1468, 2000; Lee et al., Cancer Res., 61: 2602-2608, 2001, Shipman et al. Br. J. Cancer, 98: 665-672 (1997).

The statins, such as fluvastatin (Lescol, Novartis Pharma AG) are inhibitors of the enzyme 3-hydroxy-3-methylglutaryl coenzyme A reductase, i.e. HMG-CoA reductase inhibitors, and are widely used as cholesterol lowering agents.

It has now been found that if certain types of bisphosphonates are used in combination with certain types of HMG-CoA reductase inhibitors to treat human myeloma cells in vitro, that the bisphosphonate and HMG-CoA reductase inhibitor act synergistically to inhibit myeloma cell proliferation and induce myeloma cell apopotosis. Additionally it has been found that the HMG-CoA reductase inhibitor fluvastatin on its own inhibits proliferation and induces apoptosis of human myeloma cells in vitro.

Accordingly the present invention provides a pharmaceutical composition for treatment of malignancies, which comprises in combination a bisphosphonate and an HMG-CoA reductase inhibitor for simultaneous, sequential or separate use.

Further the invention provides the use of an HMG-CoA reductase inhibitor for the preparation of a medicament, for use in combination with a bisphosphonate for treatment of a malignant disease.

In the alternative the invention provides use of a bisphosphonate for the preparation of a medicament for use in combination with an HMG-CoA reductase inhibitor for treatment of a malignant disease.

In a further aspect the invention provides a method of treating a patient suffering from a malignant disease comprising administering to the patient an effective amount of a bisphosphonate and an effective amount of an HMG-CoA reductase inhibitor.

Yet further the invention provides use of an HMG-CoA reductase inhibitor in combination with a bisphosphonate to inhibit cancer cell growth or induce cancer cell apoptosis.

Accordingly also the present invention further provides a pharmaceutical composition for inhibiting cancer cell growth or inducing cancer cell apoptosis which comprises in combination a bisphosphonate and an HMG-CoA reductase inhibitor for simultaneous, sequential or separate use.

Further the invention provides the use of a bisphosphonate for the preparation of a medicament, for use in combination with an HMG-CoA reductase inhibitor for inhibiting cancer cell growth or inducing cancer cell apoptosis.

In accordance with the present invention it has been found that HMG-CoA reductase inhibitors on their own inhibit cancer cell growth or induce cancer cell apoptosis.

Thus in yet further embodiments the invention provides:

a method of treating a patient suffering from a malignant disease comprising administering to the patient an effective amount of an HMG-CoA reductase inhibitor; and
use of an HMG-CoA reductase inhibitor for the preparation of an anti-cancer medicament.

In the present description the term “treatment” includes both prophylactic or preventative treatment as well as curative or disease modifying treatment, including treatment of patients at risk of contracting the disease or suspected to have contracted the disease as well as ill patients.

The invention is generally applicable to the treatment of malignant diseases for which bisphosphonate treatment is indicated. Thus typically the disease is a malignant disease which is associated with the development of bone metastases or excessive bone resorption. Examples of such diseases include cancers, such as breast and prostate cancers, multiple myeloma (MM), tumour induced hypertension (TIH) and similar diseases and conditions. In particular the invention is applicable to the treatment of multiple myeloma (MM) and associated bone metastases (BM).

The compositions, uses and methods of the present invention represent an improvement to existing therapy of malignant diseases in which bisphosphonates are used, e.g. to prevent or inhibit development of bone metastases or excessive bone resorption, and in which bisphosphonate treatment also inhibits cancer cell growth or induces cancer cell apoptosis. The combination of a bisphosphonate with an HMG-CoA reductase inhibitor advantageously gives rise to enhanced, advantageously synergistic, levels of cancer cell growth inhibition or cancer cell apoptosis, e.g. inhibition proliferation and induction of apoptosis of human myeloma cells.

The bisphosphonates for use in the present invention are preferably N-bisphosphonates.

For the purposes of the present description an N-bisphosphonate is a compound which in addition to the characteristic geminal bisphosphate moiety comprises a nitrogen containing side chain, e.g. a compound of formula I

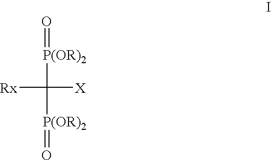

wherein
X is hydrogen, hydroxyl, amino, alkanoyl, or an amino group substituted by C₁-C₄ alkyl, or alkanoyl;
R is hydrogen or C₁-C₄ alkyl and
Rx is a side chain which contains an optionally substituted amino group, or a nitrogen containing heterocycle (including aromatic nitrogen-containing heterocycles),
and pharmaceutically acceptable salts thereof or any hydrate thereof.

Thus, for example, suitable N-bisphosphonates for use in the invention may include the following compounds or a pharmaceutically acceptable salt thereof, or any hydrate thereof: 3-amino-1-hydroxypropane-1,1-diphosphonic acid pamidronic acid), e.g. pamidronate (APD); 3-(N,N-dimethylamino)-1-hydroxypropane-1,1-diphosphonic acid, e.g. dimethyl-APD; 4-amino-1-hydroxybutane-1,1-diphosphonic acid (alendronic acid), e.g. alendronate; 1-hydroxy-3-(methylpentylamino)-propylidene-bisphosphonic acid, ibandronic acid, e.g. ibandronate; 6-amino-1-hydroxyhexane-1,1-diphosphonic acid, e.g. amino-hexyl-BP; 3-(N-methyl-N-n-pentylamino)-1-hydroxypropane-1,1-diphosphonic acid, e.g. methyl-pentyl-APD (=BM 21.0955); 1-hydroxy-2-(imidazol-1-yl)ethane-1,1-diphosphonic acid, e.g. zoledronic acid; 1-hydroxy-2-(3-pyridyl)ethane-1,1-diphosphonic acid (risedronic acid), e.g. risedronate, including N-methylpyridinium salts thereof, for example N-methylpyridinium iodides such as NE-10244 or NE-10446; 3-[N-(2-phenylthioethyl)-N-methylamino]-1-hydroxypropane-1,1-diphosphonic acid; 1-hydroxy-3-(pyrrolidin-1-yl)propane-1,1-diphosphonic acid, e.g. EB 1053 (Leo); 1-(N-phenylaminothiocarbonyl)methane-1,1-diphosphonic acid, e.g. FR 78844 (Fujisawa); 5-benzoyl-3,4-dihydro-2H-pyrazole-3,3-diphosphonic acid tetraethyl ester, e.g. U-81581 (Upjohn); and 1-hydroxy-2-(imidazo[1,2-a]pyridin-3-yl)ethane-1,1-diphosphonic acid, e.g. YM 529.

In one embodiment a particularly preferred N-bisphosphonate for use in the invention comprises a compound of Formula II

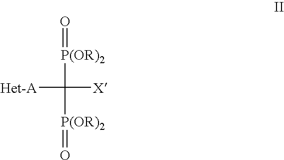

wherein
Het is an imidazole, oxazole, isoxazole, oxadiazole, thiazole, thiadiazole, pyridine, 1,2,3-triazole, 1,2,4-triazole or benzimidazole radical, which is optionally substituted by alkyl, alkoxy, halogen, hydroxyl, carboxyl, an amino group optionally substituted by alkyl or alkanoyl radicals or a benzyl radical optionally substituted by alkyl, nitro, amino or aminoalkyl;
A is a straight-chained or branched, saturated or unsaturated hydrocarbon moiety containing from 1 to 8 carbon atoms:
X′ is a hydrogen atom, optionally substituted by alkanoyl, or an amino group optionally substituted by alkyl or alkanoyl radicals, and
R is a hydrogen atom or an alkyl radical,
   and the pharmacologically acceptable salts thereof

In a further embodiment a particularly preferred bisphosphonate for use in the invention comprises a compound of Formula III

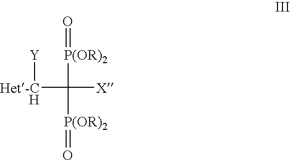

wherein
Het′ is a substituted or unsubstituted heteroaromatic five-membered ring selected from the group consisting of imidazolyl, imidazolinyl, isoxazolyl, oxazolyl, oxazolinyl, thiazolyl, thiazolinyl, triazolyl, oxadiazolyl and thiadiazolyl wherein said ring can be partly hydrogenated and wherein said substituents are selected from at least one of the group consisting of C₁-C₄ alkyl, C₁-C₄ alkoxy, phenyl, cyclohexyl, cyclohexylmethyl, halogen and amino and wherein two adjacent alkyl substituents of Het can together form a second ring;
Y is hydrogen or C₁-C₄ alkyl;
X″ is hydrogen, hydroxyl, amino, or an amino group substituted by C₁-C₄ alkyl, and
R is hydrogen or C₁-C₄ alkyl;
   as well as the pharmacologically acceptable salts and isomers thereof.

In a yet further embodiment a particularly preferred bisphosphonate for use in the invention comprises a compound of Formula IV

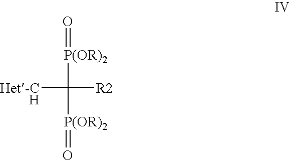

wherein
Het′″ is an imidazolyl, 2H-1,2,3-, 1H-1,2,4- or 4H-1,2,4-triazolyl, tetrazolyl, oxazolyl, isoxazolyl, oxadiazolyl, thiazolyl or thiadiazolyl radical which is unsubstituted or C-mono- or di-substituted by lower alkyl, by lower alkoxy, bx phenyl which may in turn be mono- or disubstituted by lower alkyl, lower alkoxy and/or halogen, by hydroxy, by di-lower alkylamino, by lower alkylthio and/or by halogen and is N-substituted at a substitutable N-atom by lower alkyl or by phenyl-lower alkyl which may in turn be mono- or di-substituted in the phenyl moiety by lower alkyl, lower alkoxy and/or halogen, and
R2 is hydrogen, hydroxy, amino, lower alkylthio or halogen,
lower radicals having up to and including 7 C-atoms,
   or a pharmacologically acceptable salt thereof.

Examples of particularly preferred N-bisphosphonates for use in the invention are:
2-(1-Methylimidazol-2-yl)-1-hydroxyethane-1,1-diphosphonic acid;
2-(1-Benzylimidazol-2-yl)-1-hydroxyethane-1,1-diphosphonic acid;
2-(1-Methylimidazol-4-yl)-1-hydroxyethane-1,1-diphosphonic acid;
1-Amino-2-(1-methylimidazol-4-yl)ethane-1,1-diphosphonic acid;
1-Amino-2-(1-benzylimidazol-4-yl)ethane-1,1-diphosphonic acid;
2-(1-Methylimidazol-2-yl)ethane-1,1-diphosphonic acid;
2-(1-Benzylimidazol-2-yl)ethane-1,1-diphosphonic acid;
2-(Imidazol-1-yl)-1-hydroxyethane-1,1-diphosphonic acid;
2-(Imidazol-1-yl)ethane-1,1-diphosphonic acid;
2-(4H-1,2,4-triazol-4-yl)-1-hydroxyethane-1,1-diphosphonic acid;
2-(Thiazol-2-yl)ethane-1,1-diphosphonic acid;
2-(Imidazol-2-yl)ethane-1,1-diphosphonic acid;
2-(2-Methylimidazol-4(5)-yl)ethane-1,1-diphosphonic acid;
2-(2-Phenylimidazol-4(5)-yl)ethane-1,1-diphosphonic acid;
2-(4,5-Dimethylimidazol-1-yl)-1-hydroxyethane-1,1-diphosphonic acid, and
2-(2-Methylimidazol-4(5)-yl)-1-hydroxyethane-1,1-diphosphonic acid,
   and pharmacologically acceptable salts thereof.

The most preferred N-bisphosphonate for use in the invention is 2-(imidazol-1yl)-1-hydroxyethane-1,1-diphosphonic acid (zoledronic acid) or a pharmacologically acceptable salt thereof.

All the N-bisphosphonic acid derivatives mentioned above are well known from the literature. This includes their manufacture (see e.g. EP-A-513760, pp. 13-48). For example, 3-amino-1-hydroxypropane-1,1-diphosphonic acid is prepared as described e.g. in U.S. Pat. No. 3,962,432 as well as the disodium salt as in U.S. Pat. Nos. 4,639,338 and 4,711,880, and 1-hy-droxy-2-(imidazol-1-yl)ethane-1,1-diphosphonic acid is prepared as described e.g. in U.S. Pat. No. 4,939,130. See also U.S. Pat. Nos. 4,777,163 and 4,687,767.

The N-bisphosphonates may be used in the form of an isomer or of a mixture of isomers where appropriate, typically as optical isomers such as enantiomers or diastereoisomers or geometric isomers, typically cis-trans isomers. The optical isomers are obtained in the form of the pure antipodes and/or as racemates.

The N-bisphosphonates can also be used in the form of their hydrates or include other solvents used for their crystallisation.

The HMG-CoA reductase inhibitors used in the pharmaceutical compositions and treatment methods of the present invention are preferably statins, including for example, atorvastatin, cerivastatin, nisvastatin, pitavastatin, pravastatin, simavastatin, fluvastatin and similar compounds and salts and esters thereof.

In particular the HMG-CoA reductase inhibitor is fluvastatin or a related compound, such as the HMG-CoA reductase inhibitors described in EP 0 114 027B, U.S. Pat. No. 4,739,073 and U.S. Pat. No. 5,354,772, and pharmaceutically acceptable salts and esters thereof.

Pharmacologically acceptable salts of bisphosphonates and HMG-CoA reductase inhibitors are preferably salts with bases, conveniently metal salts derived from groups Ia, Ib, IIa and IIb of the Periodic Table of the Elements, including alkali metal salts, e.g. potassium and especially sodium salts, or alkaline earth metal salts, preferably calcium or magnesium salts, and also ammonium salts with ammonia or organic amines.

Especially preferred pharmaceutically acceptable salts of the N-bisphosphonates are those where one, two, three or four, in particular one or two, of the acidic hydrogens of the bisphosphonic acid are replaced by a pharmaceutically acceptable cation, in particular sodium, potassium or ammonium, in first instance sodium.

A very preferred group of pharmaceutically acceptable salts of the N-bisphosphonates is characterized by having one acidic hydrogen and one pharmaceutically acceptable cation, especially sodium, in each of the phosphonic acid groups.

The Agents of the Invention, i.e. the HMG-CoA reductase inhibitor and the bisphosphonate are preferably used in the form of pharmaceutical preparations that contain the relevant therapeutically effective amount of each active ingredient (either separately or in combination) optionally together with or in admixture with inorganic or organic, solid or liquid, pharmaceutically acceptable carriers which are suitable for administration. The HMG-CoA REDUCTASE inhibitor and bisphosphonate active ingredients may be present in the same pharmaceutical compositions, e.g. as a fixed combinations, though are preferably in separate pharmaceutical compositions. Thus the active ingredients may be administered at the same time (e.g. simultaneously) or at different times (e.g. sequentially) and over different periods of time, which may be separate from one another or overlapping.

The N-bisphosphonates are preferably used in the form of pharmaceutical compositions that contain a therapeutically effective amount of active ingredient optionally together with or in admixture with inorganic or organic, solid or liquid, pharmaceutically acceptable carriers which are suitable for administration.

The N-bisphosphonate pharmaceutical compositions may be, for example, compositions for enteral, such as oral, rectal, aerosol inhalation or nasal administration, compositions for parenteral, such as intravenous or subcutaneous administration, or compositions for transdermal administration (e.g. passive or iontophoretic).

Preferably, the N-bisphosphonate pharmaceutical compositions are adapted to oral or parenteral (especially intravenous, intra-arterial or transdermal) administration. Intravenous and oral, first and foremost intravenous, administration is considered to be of particular importance. Preferably the N-bisphosphonate active ingredient is in a parenteral form, most preferably an intravenous form.

The particular mode of administration and the dosage may be selected by the attending physician taking into account the particulars of the patient, especially age, weight, life style, activity level, and disease state as appropriate. Most preferably, however, the N-bisphosphonate is administered intravenously.

The dosage of the N-bisphosphonate for use in the invention may depend on various factors, such as effectiveness and duration of action of the active ingredient, mode of administration, warm-blooded species, and/or sex, age, weight and individual condition of the warn-blooded animal.

Normally the dosage is such that a single dose of the bisphosphonate active ingredient from 0.002-20.0 mg/kg, especially 0.01-10.0 mg/kg, is administered to a warm-blooded animal weighing approximately 75 kg. If desired, this dose may also be taken in several, optionally equal, partial doses.

“mg/kg” means mg drug per kg body weight of the mammal—including man—to be treated.

The HMG-CoA reductase pharmaceutical compositions may be, for example, compositions for enteral, such as oral, rectal, aerosol inhalation or nasal administration, compositions for parenteral, such as intravenous or subcutaneous administration, or compositions for transdermal administration (e.g. passive or iontophoretic).

Preferably, the HMG-CoA reductase pharmaceutical compositions are adapted to oral or parenteral (especially oral) administration. Preferably the HMG-CoA reductase inhibitor active ingredient is in oral form.

The particular mode of administration and the dosage may be selected by the attending physician taking into account the particulars of the patient, especially age, weight, life style, activity level, etc.

The dosage of the Agents of the Invention may depend on various factors, such as effectiveness and duration of action of the active ingredient, mode of administration, warm-blooded species, and/or sex, age, weight and individual condition of the warm-blooded animal.

The pharmacologically active compounds of the invention are useful in the manufacture of pharmaceutical compositions comprising an effective amount thereof in conjunction or admixture with excipients or carriers suitable for either enteral or parenteral application. Preferred are tablets and gelatin capsules comprising the active ingredient together with a) diluents, e.g. lactose, dextrose, sucrose, mannitol, sorbitol, cellulose and/or glycine; b) lubricants, e.g. silica, talcum, stearic acid, its magnesium or calcium salt and/or polyethyleneglycol; for tablets also c) binders e.g. magnesium aluminum silicate, starch paste, gelatin, tragacanth, methylcellulose, sodium carboxymethylcellulose and or polyvinylpyrrolidone; if desired d) disintegrants, e.g. starches, agar, alginic acid or its sodium salt, or effervescent mixtures; and/or e) absorbents, colorants, flavors and sweeteners. Injectable compositions are preferably aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1 to 75%, preferably about 1 to 50%, of the active ingredient.

Tablets may be either film coated or enteric coated according to methods known in the art.

Suitable formulations for transdermal application include an effective amount of a compound of the invention with carrier. Advantageous carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable formulations for topical application, e.g. to the skin and eyes, include aqueous solutions, suspensions, ointments, creams, gels or sprayable formulations, for example, for delivery by aerosol or the like. Such topical delivery systems will in particular be appropriate for dermal application, e.g. for the treatment of skin cancer, for example, for prophylactic use in creams, lotions sprays and the like

The dosage of HMG-CoA reductase inhibitor administered is dependent on the species of warm-blooded animal (mammal), the body weight, age and individual condition, and on the form of administration. A unit dosage for oral administration to a mammal of about 50 to 70 kg may contain between about 5 and 1000 mg, e.g. from 100-800 mg, preferably 50-200 mg of the active ingredient.

HMG-CoA reductase inhibitor formulations in single dose unit form contain preferably from about 1% to about 90%, and formulations not in single dose unit form contain preferably from about 0.1% to about 50%, of the active ingredient. Single dose unit forms such as capsules, tablets or dragées contain e.g. from about 1 mg to about 1000 mg of the active ingredient.

HMG-CoA reductase inhibitor pharmaceutical preparations for enteral and parenteral administration are, for example, those in dosage unit forms, such as dragées, tablets or capsules and also ampoules. They are prepared in a manner known per se, for example by means of conventional mixing, granulating, confectioning, dissolving or lyophilising processes. For example, pharmaceutical preparations for oral administration can be obtained by combining the active ingredient with solid carriers, where appropriate granulating a resulting mixture, and processing the mixture or granulate, if desired or necessary after the addition of suitable adjuncts, into tablets or dragée cores.

Preferred formulations for the HMG-CoA reductase inhibitors are described in GB 2,262,229A and U.S. Pat. No. 5,356,896.

Other orally administrable pharmaceutical preparations are dry-filled capsules made of gelatin, and also soft, sealed capsules made of gelatin and a plasticiser, such as glycerol or sorbitol. The dry-filled capsules may contain the active ingredient in the form of a granulate, for example in admixture with fillers, such as lactose, binders, such as starches, and/or glidants, such as talc or magnesium stearate, and, where appropriate, stabilisers. In soft capsules the active ingredient is preferably dissolved or suspended in suitable liquids, such as fatty oils, paraffin oil or liquid polyethylene glycols, it being possible also for stabilisers to be added.

Parenteral formulations are especially injectable fluids that are effective in various manners, such as intravenously, intramuscularly, intraperitoneally, intranasally, intradermally or subcutaneously. Such fluids are preferably isotonic aqueous solutions or suspensions which can be prepared before use, for example from lyophilised preparations which contain the active ingredient alone or together with a pharmaceutically acceptable carrier. The pharmaceutical preparations may be sterilised and/or contain adjuncts, for example preservatives, stabilisers, wetting agents and/or emulsifiers, solubilisers, salts for regulating the osmotic pressure and/or buffers.

Suitable formulations for transdermal application include an effective amount of the active ingredient with carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the active ingredient of the skin of the host at a controlled and predetermined rate over a prolonged period of time, and means to secure the device to the skin.

In preferred embodiments, in view of the synergistic activity of the bisphosphonates and HMG-CoA reductase inhibitors, lower doses of both compounds may be used than would be the case if the bisphosphonate or HMG-CoA reductase inhibitor were used as sole treatment.

The following examples are intended to illustrate the invention and are not to be construed as being limitations thereon.

### EXAMPLES

### A. Formulation Examples

### Example 1

### Wet Granulated Tablet Composition

| Amount per tablet | | Ingredient |
|---|---|---|
| 25 | mg | HMG-CoA reductase inhibitor |
| 79.7 | mg | Macrocrystalline cellulose |
| 79.7 | mg | Lactose monohydrate |
| 6 | mg | Hydroxypropyl cellulose |
| 8 | mg | Croscarmellose sodium |
| 0.6 | mg | Iron oxide |
| 1 | mg | Magnesium stearate |

Tablet dose strengths of between 5 and 125 mg can be accommodated by varying total weight, and the ratio of the first three ingredients. Generally it is preferable to maintain a 1:1 ratio for microcrystalline cellulose: lactose monohydrate.

### Example 2

### Directly Compressed Tablet Composition

| Amount per tablet | | Ingredient |
|---|---|---|
| 25 | mg | HMG-CoA reductase inhibitor |
| 106.9 | mg | Microcrystalline cellulose |
| 106.9 | mg | Lactose anhydrate |
| 7.5 | mg | Croscarmellose sodium |
| 3.7 | mg | Magnesium stearate |

Tablet dose strengths of between 5 and 125 mg can be accommodated by varying total tablet weight, and the ratio of the first three ingredients. Generally it is preferable to maintain a 1:1 ratio for microcrystalline cellulose:lactose monohydrate.

### Example 3

### Hard Gelatine Capsule Composition

| Amount per capsule | | Ingredient |
|---|---|---|
| 25 | mg | HMG-CoA reductase inhibitor |
| 37 | mg | Macrocrystalline cellulose |
| 37 | mg | Lactose anhydrate |
| 1 | mg | Magnesium stearate |
| 1 | capsule | Hard gelatin capsule |

Capsule dose strengths of between 1 and 50 mg can be accommodated by varying total fill weight, and the ratio of the first three ingredients. Generally it is preferable to maintain a 1:1 ratio for microcrystalline cellulose:lactose monohydrate.

### Example 4

### Oral Solution

| Amount per 5 mL | Ingredient |
|---|---|
| 50 mg | HMG-CoA reductase inhibitor |
| to 5 mL | with Polyethylene oxide 400 |

### Example 5

### Oral Suspension

| Amount per 5 mL dose | Ingredient |
|---|---|
| 101 mg | HMG-CoA reductase inhibitor |
| 150 mg | Polyvinylpyrrolidone |

### Oral Suspension

| Amount per 5 mL dose | | Ingredient |
|---|---|---|
| 2.5 | mg | Poly oxyethylene sorbitan monolaurate |
| 10 | mg | Benzoic acid |

| | |
|---|---|
| to 5 mL | with sorbitol solution (70%) |

Suspension dose strengths of between 1 and 50 mg/5 ml can be accommodated by varying the ratio of the first two ingredients.

### Example 6

### Intravenous Infusion

| Amount per 200 mL dose | | Ingredient |
|---|---|---|
| 1 | mg | HMG-CoA reductase inhibitor |
| 0.2 | mg | Polyethylene oxide 400 |
| 1.8 | mg | Sodium chloride |

| | |
|---|---|
| to 200 mL | Purified water |

### Example 7

Capsules containing coated pellets of active ingredient, for example, disodium pamidronate pentahydrate, as active ingredient:

Core pellet:

| | | |
|---|---|---|
| active ingredient (ground) | 197.3 | mg |
| Microcrystalline cellulose | 52.7 | mg |
| (Avicel ® PH 105) | 250.0 | mg |

+Inner coating:

| | | |
|---|---|---|
| Cellulose HP-M 603 | 10.0 | mg |
| Polyethylene glycol | 2.0 | mg |
| Talc | 8.0 | mg |
| | 270.0 | mg |

+Gastric juice-resistant outer coating:

| | | |
|---|---|---|
| Eudragit ® L 30 D (solid) | 90.0 | mg |
| Triethyl citrate | 21.0 | mg |
| Antifoam ® AF | 2.0 | mg |
| Water | | |
| Talc | 7.0 | mg |
| | 390.0 | mg |

A mixture of disodium pamidronate with Avicel® PH 105 is moistened with water and kneaded, extruded and formed into spheres. The dried pellets are then successively coated in the fluidized bed with an inner coating, consisting of cellulose HP-M 603, polyethylene glycol (PEG) 8000 and talc, and the aqueous gastric juice-resistant coat, consisting of Eudragit® L 30 D, triethyl citrate and Antifoam® AF. The coated pellets are powdered with talc and filled into capsules (capsule size 0) by means of a commercial capsule filling machine, for example Höfliger and Karg.

### Example 8

Monolith adhesive transdermal system, containing as active ingredient, for example, 1-hydroxy-2-(imidazol-1-yl)-ethane-1,1-diphosphonic acid:

### Composition:

| | | |
|---|---|---|
| polyisobutylene (PIB) 300 | 5.0 | g |
| (Oppanol B1, BASF) | | |
| PIB 35000 | 3.0 | g |
| (Oppanol B10, BASF) | | |
| PIB 1200000 | 9.0 | g |
| (Oppanol B100, BASF) | | |
| hydrogenated hydrocarbon resin | 43.0 | g |
| (Escorez 5320, Exxon) | | |
| 1-dodecylazacycloheptan-2-one | 20.0 | g |
| (Azone, Nelson Res., Irvine/CA) | | |
| active ingredient | 20.0 | g |
| Total | 100.0 | g |

### Preparation:

The above components are together dissolved in 150 g of special boiling point petroleum fraction 100-125 by rolling on a roller gear bed. The solution is applied to a polyester film (Hostaphan, Kalle) by means of a spreading device using a 300 mm doctor blade, giving a coating of about 75 g/m². After drying (15 minutes at 60° C.), a silicone-treated polyester film (thickness 75 mm, Laufenberg) is applied as the peel-off film. The finished systems are punched out in sizes in the wanted form of from 5 to 30 cm² using a punching tool. The complete systems are sealed individually in sachets of aluminised paper.

### Example 9

Vial containing 1.0 mg dry, lyophilized 1-hydroxy-2-(imidazol-1-yl)ethane-1,1-diphosphonic acid (mixed sodium salts thereof). After dilution with 1 ml of water, a solution (concentration 1 mg/ml) for i.v. infusion is obtained.

### Composition:

| | | |
|---|---|---|
| active ingredient (free diphosphonic acid) | 1.0 | mg |
| mannitol | 46.0 | mg |
| Trisodium citrate × 2 H₂O | ca. 3.0 | mg |
| water | 1 | ml |
| water for injection | 1 | ml. |

In 1 ml of water, the active ingredient is titrated with trisodium citrate×2H₂O to pH 6.0. Then, the mannitol is added and the solution is lyophilized and the lyophilisate filled into a vial.

### Example 10

Ampoule containing active ingredient, for instance disodium pamidronate pentahydrate dissolved in water. The solution (concentration 3 mg/ml) is for i.v. infusion after dilution.

### Composition:

| | | |
|---|---|---|
| active ingredient | 19.73 | mg |
| ( [symbol] 5.0 mg of anhydrous active ingredient) | | |
| mannitol | 250 | mg |
| water for injection | 5 | ml. |

### Example 11

In vitro analysis of growth inhibition and apoptosis induction in human myeloma cell lines by the 3′-hydroxy-3-methylglutaryl coenzyme A (HMG-CoA) reductase inhibitor fluvastatin alone and in combination with Zometa® (zoledronic acid)

We investigated the cytotoxic effect of the HMG-CoA reductase inhibitor fluvastatin on the human multiple myeloma cell lines LP-1, OPM-2, U266, NCI-H929 and RPMI-8226 in vitro using a tetrazolium reduction assay. After 3 days culture in the presence of 0 to 50 μM fluvastatin, the Promega MTS assay reagent was used to determine the level of inhibition of cell proliferation and/or cell death Fluvastatin concentrations as low as 2.5 μM significantly inhibited proliferation of all cell lines except RPMI-8226 (p<0.05 by paired Student's t-test). Concentrations of 25 μM and 50 μM significantly inhibited proliferation in all cell lines (p<0.05 by paired Student's t-test), with inhibition at 50 μM ranging from 45 to >90% for U266 to OPM-2.

Using the same assay we investigated whether the activity of fluvastatin against multiple myeloma in vitro could be enhanced by the addition of the bisphosphonate Zometa® (zoledronic acid). Using 80% cell inhibition as an end point, isobolograms were constructed to visualize the interaction between fluvastatin and Zometa®. Isobologram analysis indicated that fluvastatin and Zometa® synergistically to induce cell death in human myeloma cell lines. To illustrate this point, >50 μM fluvastatin or <100 μM Zometa® alone was required to induce 80% cell death in the myeloma cell line LP-1 but the combination of 25 μM fluvastatin and 0.21 μM Zometa® had the same effect.

These initial data indicate that fluvastatin is a potential therapeutic agent for multiple myeloma both as a single agent and in combination with other agents such as Zometa®.

1-12. (canceled)

13. A method of treating a patient suffering from multiple myeloma comprising administering to the patient an effective amount of 2-(imidazol-1yl)-1-hydroxyethane-1,1-diphosphonic acid or a pharmacologically acceptable salt thereof and an effective amount of fluvastatin.
